(19) United States
(12) Patent Application Publication
(10) Pub. No.: US 2023/0057267 A1
(43) Pub. Date: Feb. 23, 2023

(54) FERTILITY TRACKING AND STIMULATION DEVICE

(71) Applicant: Philip DAgostino, Broad Channel, NY (US)

(72) Inventor: Philip DAgostino, Broad Channel, NY (US)

(21) Appl. No.: 17/445,654

(22) Filed: Aug. 23, 2021

(51) Int. Cl. A61B 10/00; A61H 19/00

(52) U.S. Cl. CPC A61B 10/0012; A61H 19/44; A61B 2010/0019; A61B 2562/0271

(57) ABSTRACT

A fertility tracking system is disclosed. The system includes a sensual fertility device that provides stimulation to the woman while also providing feedback regarding the status of the user's menstrual cycle in the form of changing colors of the sensual fertility device. The system further includes systemic software (such as a mobile application) that communicates with the sensual fertility device to determine and instruct which color should be activated on the sensual fertility device.

### BACKGROUND OF THE INVENTION

The present invention relates to fertility tools and, more particularly, to a fertility tracking and stimulation device that provides a changing visual aesthetic indicative of the current point in a fertility window a woman is that is using the device.

Devices generally shaped like an erect penis are frequently used for sexual stimulation or to enhance sexual pleasure. These devices include dildos and vibrators and are also colloquially referred to as “sex toys”. Prior to the present invention, they were only configured for use for stimulative purposes.

Conventionally, tracking ovulation when trying to conceive a child can be a stressful endeavor. Available options include, for example, a vaginal thermometer that is used to simply take temperature is not used for pleasure and can be awkward to use. Mobile applications also exist for tracking when a woman is likely ovulating. However, those are merely based on the calendar and where in a menstrual cycle a woman likely is, which is not necessarily accurate to what is actually occurring in the woman's body. Further, like taking temperature with a vaginal thermometer, they reduce sexual experiences to clinical ones.

These other devices and systems are deficient because they provide only a single benefit (either sexual stimulation or ovulation tracking) and they result in sex becoming a clinical process. This can add further stress on a couple that may already be having trouble conceiving. As can be seen, there is a need for a device in accordance with the presently described invention.

### SUMMARY OF THE INVENTION

In one aspect of the present invention, a fertility tracking system comprises: a computing device comprising a computing device processor, a memory, a user interface, and a computing device communication interface for communicating over a network; and a plurality of program instructions stored in the memory that, when executed by the computing device processor, cause the computing device processor to: identify a menstrual cycle phase of a user; select a device color instruction based on the identified menstrual cycle phase, the device color instruction comprising a selected color; and transmit, over the network, the device color instruction; and a sensual fertility device comprising: an outer substantially cylindrical body; a color-changing light housed within the body; a sensual fertility device communication interface housed within the body; and a sensual fertility device processor housed within the body and configured to receive the device color instruction and to activate the color-changing light to display the selected color.

In another aspect of the present invention, a sensual fertility device comprises: an outer substantially cylindrical body; a color-changing light housed within the body; a sensual fertility device communication interface housed within the body; a sensual fertility device processor housed within the body and configured to: receive a device color instruction based on an identified menstrual cycle phase of a user, the device color instruction comprising a selected color; and activate the color-changing light to display the selected color.

These and other features, aspects and advantages of the present invention will become better understood with reference to the following drawings, description and claims.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 is a perspective view of a device of an embodiment of the present invention;

FIG. 2 is a front view of the device of the embodiment of the present invention;

FIG. 3 is a schematic view of the embodiment of the present invention, showing internal components of the present invention and in use with a mobile application of the present invention; and

FIG. 4 is a flow chart illustrating an exemplary method of use of the present invention.

### DETAILED DESCRIPTION OF THE INVENTION

The subject disclosure is described with reference to the drawings, wherein like reference numerals are used to refer to like elements throughout. In the following description, for purposes of explanation, numerous specific details are set forth in order to provide a thorough understanding of the present disclosure such that one skilled in the art will be enabled to make and use the present invention. It may be evident, however, that the present disclosure may be practiced without some of these specific details.

Broadly, one embodiment of the present invention is a fertility tracking and stimulation device (also referred herein as a sensual fertility device) that includes an outer substantially cylindrical phallic shaped body, a color-changing light housed within the body, a sensual fertility device communication interface housed within the body, and a sensual fertility device processor housed within the body. The sensual fertility device processor is designed to (1) receive a device color instruction based on an identified menstrual cycle phase of a user, the device color instruction comprising a selected color, and (2) activate the color-changing light to display the selected color.

More specifically, the sensual fertility device glows with changing colors (e.g., blue to purple to red) based on the current point within the fertility window of the woman using it. When used as a stimulating device (i.e., “sex toy”), it will be fun and pleasurable to use. Further, the visual stimulus for fertility (i.e., its additional use as a physiological measurement device) may enhance the excitement while removing the clinical feelings of family planning. The present invention is vastly superior to conventional methods of tracking ovulation because it is a sex toy, not a clinical tool. It is designed to make family planning simple, fun, and intuitive. While the present disclosure primarily describes use of the device in the context of conceiving a child, it will be appreciated that the device may also be used to indicate times of the month when extra precautions should be taken (in scenarios a woman does not wish to become pregnant) and may also be used to track the likely start of periods.

In accordance with certain aspects of the present invention, a user simply turns on the device, and it is immediately usable for various purposes, such as: (1) for sexual stimulation, (2) to conceive a baby, (3) to avoid fertility windows when the user does not wish to get pregnant. When turned on, the device emits a color based upon either physiological measurements or based on the calculated phase of the user's menstrual cycle. The color the device glows serves as a visual indicator as to what phase of a menstrual cycle the user is in.

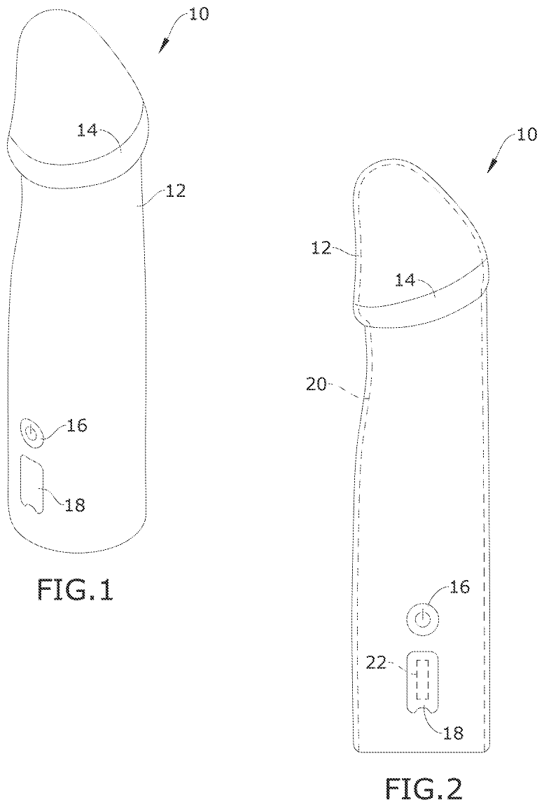

Referring to FIGS. 1-4, a fertility tracking and stimulation device 10 may include, on an outer portion thereof, an outer sleeve 12, a thermal sensor 14 (formed as, for example, a metallic ridge), a power button 16, and a charging port door 18. In certain embodiments, the outer sleeve 12 may be a transparent or translucent soft material, such as a silicone material. An internal housing 20 (also formed from a transparent or translucent material) is illustrated in FIG. 2 in dashed lines and serves to generally define the shape of the device 10. It will be appreciated by those with skill in the art that, in certain embodiments, the outer sleeve 12 of soft material may be omitted where a hard outer shell is employed. In combination, the outer sleeve 12 and internal housing form a main body (which may be phallic shaped, as shown in FIG. 1) that houses various internal components described in greater detail below. The power button 16, when pressed, powers the device 10 on and off. As shown in FIG. 2, the charging port door 18 selectively covers a charging port 22 for providing power to a rechargeable battery (not shown).

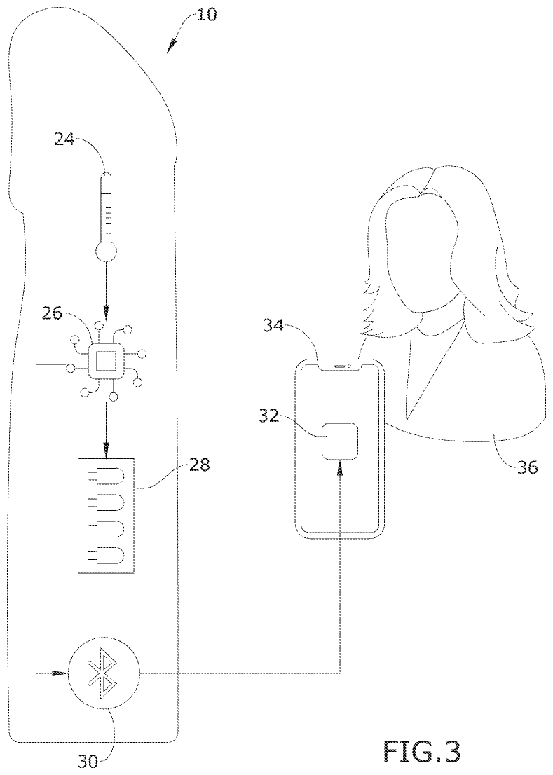

The thermal sensor 14 is disposed proximal a tip portion of the device 10 and may be, in certain embodiments, the metallic contact temperature sensor of a thermometer 24 for measuring the temperature of the inner walls of the vagina of a user 36. However, other types of thermal sensors may be used. In the illustrated embodiment, the thermometer 24 is coupled to a processor 26 (e.g., an integrated circuit), which is operable to perform a plurality of functions. For example, the processor 26 is capable of receiving temperature readings from the thermometer 24. The processor 26 is also operable to selectively activate color-changing light emitting diode (LED) lights 28 (which are embedded within the internal housing 20 of the device 10) to a preset color based upon the temperature reading, as will be described in greater detail below. As shown in FIG. 3, the device 10 may further integrate a short-range wireless chip 30 (which functions as a communication interface), such as a BLUETOOTH™ chip. The chip 30 is contained within the internal housing 20 and allows for bi-directional communication between the device 10 and systemic software (e.g., mobile application 32) loaded on a computing device, such as a mobile device 34, of the user 36.

The mobile device 34 of the present invention includes at least a processing unit coupled to a form of memory and a user interface (e.g., a graphical user interface) for providing inputs to and viewing of the mobile application 32. It will be appreciated that the computing device may include, but is not limited to, a microprocessor, a server, a desktop, laptop, and smart device, such as, a tablet and smartphone. The computing device includes a program product including a machine-readable program code/instructions for causing, when executed, the computer to perform steps. The program product may include the systemic software, which may either be loaded onto the computing device or accessed by the computing device. As mentioned above, the loaded systemic software may include an application 32 on a mobile device 34. The systemic software may also be accessed by the computing device using a web browser. The computer may access the systemic software via the web browser using the internet, extranet, intranet, host server, internet cloud and the like.

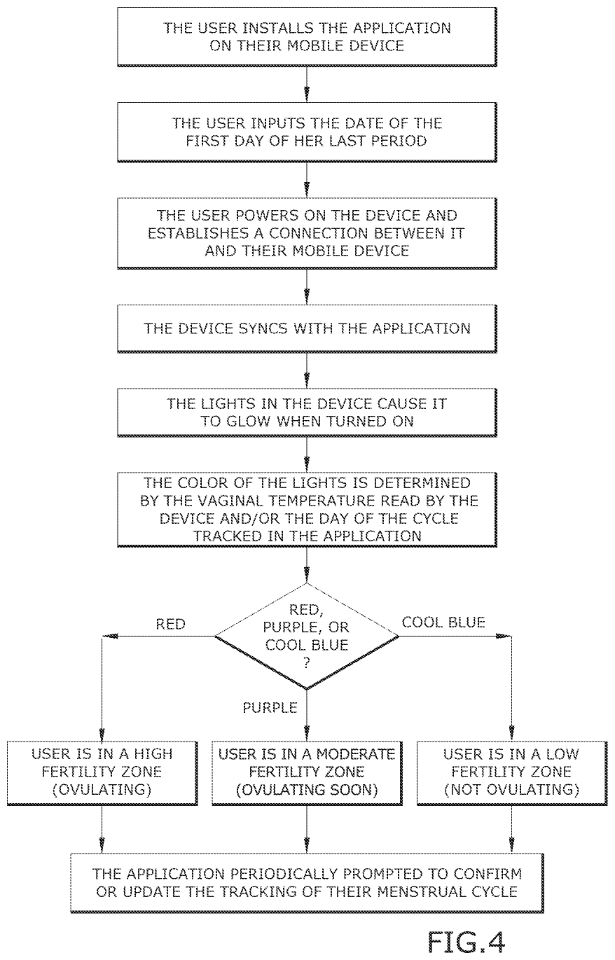

In use, the mobile application 32 provides various functionalities in combination with the device 10. FIG. 4 is an exemplary flow chart illustrative of such functionalities achieved by this pairing of application 32 and device 10. First, the application 32 may be loaded onto the mobile device 34 of the user 36. The user 36 may be prompted to input the date of the first day of her last period. The application 32 can thus thereafter track fertility cycles based upon a calendar method mode provided in the application 32. There is also provided a temperature tracking mode, which will be described in greater detail below. The device 10, after being powered on, may be automatically synced/paired with the application 32. Syncing may occur in a number of ways, such as (but not limited to) every time the device 10 is turned on, periodically, or manually through the application 32. When paired with the device 10, the application 32 may be contacted passively to gather updated data on, for example, the date of the last menstrual period. Updates within the application 32 may be made by the user, with the device 10 automatically updating its internal memory when powered on. Thus, after initial setup, all communication between the device 10 and the application 32 may occur in the background.

In embodiments where the calendar method mode is used to identify (synonymous with other terms, such as “determine” and “calculate”) at what point of the menstrual cycle the user 36 is at, the mobile application 32 is operable to track the user's cycle according to the calendar and to communicate to the processor 26 what color light 28 should be displayed. For example, red may be indicative that the user 36 is ovulating, purple may be indicative of ovulating soon, and blue may be indicative that the user 36 is not ovulating. It will be appreciated that other color combinations may be used in accordance with the present invention. Due to the transparent/translucent materials used (as previously discussed with respect to the inner housing 20 and outer sleeve 12), the device 10 effectively “glows” the appropriate color when activated and paired with the mobile application 32.

In a similar manner, a temperature tracking mode may be enabled within the application 32. When this mode is enabled, the mobile application 32 stores temperature data received from the device 10 via the wireless connection. The mobile application 32 tracks slight variations in temperature readings from the thermometer 24, which is indicative of where in a menstrual cycle the user 36 is. Thus, the point at which the user 36 is in her menstrual cycle can be determined/calculated/identified. For best results with this method, the device 10 should be used daily (ideally around the same time) to accurately track temperature fluctuations. Like the calendar method, the mobile application 32 (after recording and processing the temperature readings/data from the device 10) communicates to the processor 26 what color light 28 should be displayed on the device 10. Thus, the user 36 (and, potentially, her partner) is/are provided with visual feedback regarding her fertility status in a non-clinical, convenient way. The application 32 may also provide periodic prompts to the user 36 to confirm or update the tracking of her menstrual cycle. Referring to the sixth box of FIG. 4, will be appreciated that, in certain embodiments, rather than using a calendar method or a temperature tracking method, an algorithm may be employed to incorporate both the date and sensor data to provide greater accuracy in terms of the calculation of the menstrual cycle phase.

To summarize, a user may utilize this device 10 prior to having sexual intercourse (or as so desired, or at a specific time of day). By using the device prior sex, the is more likely to be focused on the pleasure of intercourse and of using the device 10, rather than fixating on getting pregnant. As described above, the color of the device 10 provides visual feedback to the user and may be concurrently with the stimulative use of the device 10. The present invention would not only make the person more likely to have more intercourse, and increase likelihood of pregnancy, but would also relieve the stress associated therewith, which could very well inadvertently be a secondary barrier to conception.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein.

Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The computer-based systemic software program (e.g., the mobile application) described above is for purposes of example only, and may be implemented in any type of computer system or programming or processing environment, or in a computer program, alone or in conjunction with hardware. The present invention may also be implemented in software stored on a computer-readable medium and executed as a computer program on a general purpose or special purpose computer. For clarity, only those aspects of the system germane to the invention are described, and product details well known in the art are omitted. For the same reason, the computer hardware is not described in further detail. It should thus be understood that the invention is not limited to any specific computer language, program, or computer. It is further contemplated that the mobile application of the present invention may be run on a stand-alone computer system, or may be run from a server computer system that can be accessed by a plurality of client computer systems interconnected over an intranet network, or that is accessible to clients over the Internet. To the extent the present application discloses a system, the method implemented by that system, as well as software stored on a computer-readable medium and executed as a computer program to perform the method on a general purpose or special purpose computer (e.g., a smartphone), are within the scope of the present invention. Further, to the extent the present application discloses a method, a system of apparatuses configured to implement the method are within the scope of the present invention.

What is claimed is:

1. A fertility tracking system comprising:
a computing device comprising a computing device processor, a memory, a user interface, and a computing device communication interface for communicating over a network;
a plurality of program instructions stored in the memory that, when executed by the computing device processor, cause the computing device processor to:
identify a menstrual cycle phase of a user;
select a device color instruction based on the identified menstrual cycle phase, the device color instruction comprising a selected color; and
transmit, over the network, the device color instruction; and
a sensual fertility device comprising:
an outer substantially cylindrical body;
a color-changing light housed within the body;
a sensual fertility device communication interface housed within the body; and
a sensual fertility device processor housed within the body and configured to receive the device color instruction and to activate the color-changing light to display the selected color.

2. The fertility tracking system of claim 1, wherein the sensual fertility device further comprises a temperature sensor configured to detect a body temperature of the user.

3. The fertility tracking system of claim 2, wherein the detected body temperature is transmittable to the computing device over the network, and the menstrual cycle phase is at least partially determined by using the detected body temperature.

4. The fertility tracking system of claim 1, wherein the user interface is configured to receive a date input by the user, the date being a first day of a last period of the user.

5. The fertility tracking system of claim 1, wherein the color-changing light comprises three distinct colors that are, respectively, representative of: the user ovulating, the user being close to ovulating, and the user not ovulating.

6. A sensual fertility device comprising:
an outer substantially cylindrical body;
a color-changing light housed within the body;
a sensual fertility device communication interface housed within the body; and
a sensual fertility device processor housed within the body and configured to:
receive a device color instruction based on an identified menstrual cycle phase of a user, the device color instruction comprising a selected color; and
activate the color-changing light to display the selected color.

7. The sensual fertility device of claim 6, further comprising a temperature sensor configured to detect a body temperature of the user.

8. The sensual fertility device of claim 6, wherein the body is formed from a transparent or translucent material.

9. The sensual fertility device of claim 6, wherein the body is phallic shaped.

10. The sensual fertility device of claim 6, wherein the color-changing light comprises three distinct colors that are, respectively, representative of: the user ovulating, the user being close to ovulating, and the user not ovulating.
